(19) United States
(12) Patent Application Publication
(10) Pub. No.: US 2026/0224943 A1
(43) Pub. Date: Aug. 6, 2026

(54) SYSTEM AND METHOD FOR AI-ASSISTED EXERCISE GUIDANCE BASED ON FITNESS EQUIPMENT IDENTIFICATION

(71) Applicant: Sigmond Joseph Smith, JR., Las Vegas, NV (US)

(72) Inventor: Sigmond Joseph Smith, JR., Las Vegas, NV (US)

(21) Appl. No.: 19/631,244

(22) Filed: Mar. 27, 2026

Related U.S. Application Data
(60) Provisional application No. 63/807,564, filed on May 17, 2025.

(51) Int. Cl. A63B 24/00

(52) U.S. Cl. CPC A63B 24/0075; A63B 24/0062; A63B 2024/0065

(57) ABSTRACT

The present invention relates to a system incorporating a primary wearable device configured to provide personalized exercise instruction, feedback, and program customization. The primary wearable device includes a scanning module to identify fitness equipment, a processing module to retrieve related exercise routines from a database, and a tracking module to monitor user movement in real time. Users may select from multiple instructional modes such as pre-exercise tutorials, real-time guidance, and post-exercise feedback to customize their guidance experience. The system delivers audio-visual cues, recommends substitutions for exercises causing discomfort, and highlights targeted muscle groups. The primary wearable device functions independently without requiring external apps or internet connectivity and supports content projection. The primary wearable device enables immersive, adaptive fitness training in any environment, thereby serving as an AI-driven personal trainer embedded in a compact wearable form.

### FIELD OF THE INVENTION

The present invention relates to exercise and fitness technologies, and specifically, relates to a system for providing personalized exercise instruction, feedback, and program customization using a wearable digital device that employs artificial intelligence, scanning technology, and real-time motion analysis to identify fitness equipment, monitor user performance, and deliver context-aware exercise guidance and recommendations.

### BACKGROUND

Physical fitness and strength training have long relied on human supervision and fixed instructional systems, such as personal trainers, posters, and pre-programmed exercise machines. Traditionally, individuals seeking proper guidance for exercise form, selection, and safety were required to either rely on their own experience or engage a certified fitness professional. Though effective in personalized settings, these conventional approaches are not scalable, often costly, and require the physical presence and real-time availability of trained personnel.

In recent years, mobile applications and technology-based systems have emerged to partially address these limitations. These systems allow users to view instructional videos, log workout routines, and track fitness progress over time. However, such applications typically rely on manual user input, lack real-time body movement analysis, and often require the user to interact with smartphones or tablets during workouts, which is impractical and potentially hazardous setup during physically intense sessions.

Some developments have introduced camera-based systems that use computer vision and machine learning to analyze user movements and offer posture correction feedback. Notable among these are systems such as GymLens and GymLytics, which use pose estimation through smartphone cameras or webcams to guide workouts and count repetitions. Other applications, such as Lifebeam Vi, provide motivational feedback and voice coaching. While these systems demonstrate advances over static instructional models, they remain dependent on external devices, require specific positioning, and are often tied to applications requiring Wi-Fi connectivity or cloud integration for full functionality.

Prior arts include systems that integrate exercise detection with real-time recommendation engines, such as US20220296966A1, which discloses video-based machine learning models for real-time performance feedback and workout modification. KR102632408B1 describes smart glasses interacting with exercise machines to deliver performance data. However, these systems typically require complex camera setups, lack wearability in a fully mobile and integrated way, and cannot operate independently of third party platforms or external hardware.

A major limitation in these prior systems is the lack of seamless, wearable integration that allows users to obtain immediate, context-specific exercise guidance based on both the equipment in use and their current physical movements. Most prior art solutions either analyze the user without recognizing the equipment, or identify equipment without dynamic monitoring of user form and technique. Additionally, such systems often fail to provide multiple modes of guidance (e.g., pre-exercise, during-exercise, and post-exercise feedback) and lack features for real-time substitution of exercises based on discomfort or user feedback.

Moreover, the dependency of many conventional and advanced systems on internet connectivity, third-party apps, and device tethering introduces accessibility issues, particularly in gym environments where smartphone usage may be restricted or impractical. The lack of personalization, dynamic feedback, and true offline capability limits the usefulness of these systems for a wide spectrum of users, from beginners to advanced athletes.

To address these limitations, there is a need for a system for providing personalized exercise instruction, feedback, and program customization using a wearable digital device that employs artificial intelligence, scanning technology, and real-time motion analysis to identify fitness equipment, monitor user performance, and deliver context-aware exercise guidance and recommendations. There is also a need for a system that supports multiple instructional modes, such as before, during, and after exercise execution, while enabling dynamic substitution of exercises in response to user feedback such as pain, discomfort, or disinterest. Further, there is also a need for a system that offers enhanced personalization, such as name-based voice instruction, user progress tracking, warm-up recommendations, and immersive features such as projected visual displays or visor-based guidance, all while maintaining compact portability through wearable integration.

### SUMMARY OF THE INVENTION

The following presents a simplified summary of one or more embodiments of the present disclosure to provide a basic understanding of such embodiments. This summary is not an extensive overview of all contemplated embodiments and is intended to neither identify key nor critical elements of all embodiments nor delineate the scope of any or all embodiments.

The present disclosure, in one or more embodiments, relates to a system for providing personalized exercise instruction, feedback, and program customization using artificial intelligence, scanning technology, and real-time motion analysis to identify fitness equipment, monitor user performance, and deliver context-aware exercise guidance and recommendations. The system supports multiple instructional modes, such as before, during, and after exercise execution, while enabling dynamic substitution of exercises in response to user feedback such as pain, discomfort, or disinterest. The system offers enhanced personalization, such as name-based voice instruction, user progress tracking, warm-up recommendations, and immersive features such as projected visual displays or visor-based guidance, all while maintaining compact portability through wearable integration.

In one embodiment herein, the system comprises a wearable device that is configured to be worn by the user. A scanning unit is integrated with the wearable device. The scanning unit is configured to detect and identify at least one fitness equipment located within an environment. The wearable device comprises a processor that is configured to execute a plurality of modules for providing personalized exercise instruction, feedback, and content customization. The processor is in communication with the scanning unit. The plurality of modules comprises a detection module, a storage module, a processing module, a tracking module, and an output module. The processor is configured to perform artificial intelligence-based computational operations using motion sensor data and visual input data.

In one embodiment, the artificial intelligence-based computational operations include storing a plurality of exercise routines associated with respective fitness equipment and target muscle groups using the storage module, identifying fitness equipment through visual feature extraction or tag detection based on data received from the scanning unit, retrieving one or more exercise routines corresponding to the identified fitness equipment via the processing module, monitoring a user's body movement during execution of a selected exercise routine using the tracking module, processing the monitored body movement with a pose analysis model to determine the user's posture, comparing the determined posture with predefined biomechanical reference data stored in the storage unit, and generating exercise guidance as real-time corrective feedback signals including at least one of audio coaching instructions, visual exercise form correction indicators, and motion deviation alerts using the output module. The processor is further configured to perform identification, monitoring, comparison, and generation of exercise guidance in real time within the wearable device, thereby improving posture detection accuracy and reducing processing latency.

In one embodiment herein, the detection module is configured to identify one or more fitness equipment in a surrounding environment using at least one scanning unit. The scanning unit comprises, but not limited to, a visual camera, a radio-frequency identification reader, a barcode scanner, and a proximity detector, and thereof. In one embodiment herein, the storage module is configured to store a collection of exercise routines organized by categories such as equipment type, body part, training mode, and intensity level.

In one embodiment herein, the processing module is configured to retrieve multiple exercise routines from the storage module based on the identified fitness equipment and user input. The processing module is further configured to receive user input indicating issues such as discomfort, pain, and disinterest during exercise, and in response, suggest alternate exercises aligned with the same objectives. The processing module is configured to recommend an alternative exercise based on user's discomfort and disinterest during performance of the exercise routine.

In one embodiment herein, the tracking module is configured to monitor and evaluate user body movements in real time during exercise performance. The tracking module comprises one or more motion sensors include an accelerometer and a gyroscope. The motion sensors are configured to generate motion data representing body segment orientation. The processor is configured to process the motion data to determine joint angles and movement trajectories. The tracking module includes technologies such as motion sensors, visual analysis systems, and thereof. The tracking module is configured to analyze at least one of body alignment, range of motion, and exercise tempo during execution of the selected exercise routine.

In one embodiment herein, the output module is configured to provide real-time corrective feedback during performance of the exercise routine. The output module is configured to provide instructional guidance in at least one of a pre-exercise tutorial mode, a real-time coaching mode, and a post-exercise feedback mode.

In one embodiment herein, the wearable device comprises a voice interface that is enabled by the output module to deliver personalized prompts, instructions, and encouragement incorporating user-specific identifiers. In one embodiment herein, the wearable device further comprises a display interface that is enabled by the output module to present contextual information related to the selected exercise routines.

In one embodiment herein, the wearable device further comprises a control panel with a plurality of buttons configured to enable the user to select one or more instructional modes, such as tutorial playback prior to exercise, real-time voice-based guidance during exercise, and feedback delivery after exercise completion. In one embodiment herein, the wearable device further includes an initialization feature for conducting a warm-up routine selected per body part or as a full-body sequence prior to initiating primary exercises.

In one embodiment herein, the wearable device includes a projection module that is configured to display interactive visual content on a nearby surface using projection technology for enhanced user engagement. In one embodiment herein, the wearable device is configured to operate without requiring external devices, applications, and network connectivity.

In one embodiment herein, the wearable device operates without requiring an external mobile device and internet connectivity. The system comprises a nutritional recommendation module configured to generate dietary guidance based on user fitness goals and body composition analysis performed by the wearable device.

In one embodiment herein, the wearable device comprises a primary wearable device, and a secondary wearable device configured for coordinated operation.

In one embodiment herein, the secondary wearable device is wirelessly connected to the primary wearable device. The secondary wearable device is configured to display instructional visuals without requiring network connectivity and external applications. In one embodiment herein, the wearable device is configured to allow the user to create, store, and modify custom exercise routines within the storage module based on prior performed exercises and training strategies.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a block diagram of a system for AI-assisted exercise guidance based on fitness equipment identification, in accordance with embodiments of the invention.

FIG. 2 illustrates a rear perspective view of a primary wearable device of the system, in accordance with embodiments of the invention.

FIG. 3 illustrates a perspective view of the primary wearable device activated to initiate an operation of the system, in accordance with embodiments of the invention.

FIG. 4 illustrates a perspective view of the primary wearable device detecting at least one fitness equipment, in accordance with embodiments of the invention.

FIG. 5 illustrates a perspective view of the primary wearable device presenting exercise routines, choosing at least one routine, and delivering real-time visual guidance, in accordance with embodiments of the invention.

FIG. 6 illustrates a perspective view of a secondary wearable device of the system, in accordance with embodiments of the invention.

FIG. 7 illustrates a flowchart of a method for providing personalized exercise instruction using the system, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

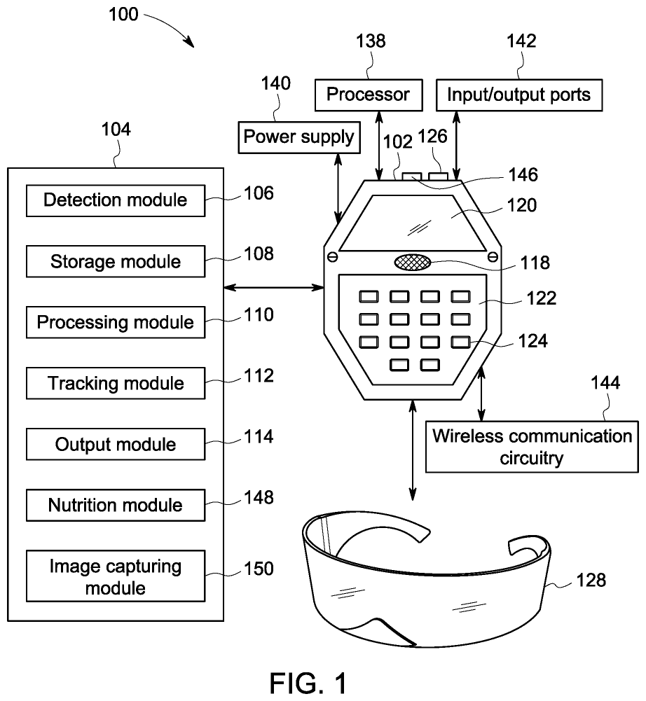

FIG. 1 refers to a block diagram of a system 100 for AI-assisted exercise guidance based on fitness equipment identification. In one embodiment herein, the system 100 provides personalized exercise instruction, feedback, and program customization using artificial intelligence, scanning technology, and real-time motion analysis to identify fitness equipment, monitor user performance, and deliver context-aware exercise guidance and recommendations. The system 100 supports multiple instructional modes, such as before, during, and after exercise execution, while enabling dynamic substitution of exercises in response to user feedback such as pain, discomfort, or disinterest. The system 100 offers enhanced personalization, such as name-based voice instruction, user progress tracking, warm-up recommendations, and immersive features such as projected visual displays or visor-based guidance, all while maintaining compact portability through wearable integration.

In certain embodiments, the system 100 improves motion tracking accuracy in dynamic fitness environments by integrating inertial sensor fusion and equipment recognition within a single wearable processing architecture. Unlike conventional fitness coaching systems that rely on external cameras or smartphone processing, the processor 138 of the wearable device 102 performs local skeletal reconstruction using fused inertial measurement unit (IMU) data and image data generated by the scanning unit. This architecture reduces computational latency and enables real-time biomechanical analysis and corrective feedback during exercise execution without reliance on external computing devices.

The integrated execution of equipment identification and biomechanical analysis within the primary wearable device 102 reduces system response latency, minimizes network dependency, and improves motion evaluation accuracy relative to externally processed fitness monitoring systems. This architecture enables continuous closed-loop feedback control based on real-time sensor acquisition, thereby improving computational efficiency and human-machine interaction responsiveness.

In certain embodiments, skeletal reconstruction is performed by mapping inertial sensor orientation data to a predefined biomechanical skeletal model comprising joint nodes representing major body joints including shoulder, elbow, hip, knee, and ankle joints.

In one embodiment herein, the system 100 comprises a primary wearable device 102 configured to execute a plurality of modules 104 that collectively facilitates personalized exercise instruction, feedback delivery, and routine customization. The modular architecture of the system 100 enables a layered and scalable approach to exercise monitoring and guidance. The plurality of modules 104 includes a detection module 106, a storage module 108, a processing module 110, a tracking module 112, and an output module 114. Each module 104 is configured to perform a specialized role in an operation of the primary wearable device 102. The integration of the plurality of modules 104 within a wearable form factor ensures mobility, hands-free usability, and a seamless fitness experience for the user.

In one embodiment, the primary wearable device 102 comprises a scanning unit 116, which is disposed on a rear surface of the primary wearable device 102.

In one embodiment, the primary wearable device 102 further comprises a processor 138 configured to execute machine learning-based classification or artificial intelligence-based computational operations. The storage module 108 configured to store exercise databases and user performance data. The term “artificial intelligence” as used herein includes machine learning models, rule-based decision engines, or statistical classification algorithms executed by the processor 138. Each of the plurality of modules 104 described herein, including the detection module 106, the storage module 108, the processing module 110, the tracking module 112, and the output module 114, may be implemented as software instructions executed by the processor 138 and stored in the storage module 108. In certain embodiments, the plurality of modules 104 may also include dedicated hardware circuitry configured to perform the corresponding operations.

The detection module 106 is configured to identify fitness equipment. The tracking module 112 is configured to monitor user movement. The scanning unit 116 is configured to detect physical gym equipment located in a surrounding environment. The primary wearable device 102 further includes a display interface 120 configured to present contextual exercise information. The output module 114 is configured to generate audio or visual feedback to the user.

In another embodiment, the processor 138 acts as the central processing unit (CPU) of the system 100, responsible for coordinating different tasks and carrying out complex operations, data processing, and decision-making by fetching instructions from the storage module 108, thereby decoding the instructions and executing the necessary actions.

In some embodiments, computational operations performed by the processing module 110 may be selectively executed locally on the processor 138 or remotely by one or more external computing systems communicatively coupled through wireless communication circuitry 144. The system 100 dynamically determines processing allocation based on latency requirements, power availability, network connectivity, or computational complexity.

In certain embodiments, anonymized performance data generated by the tracking module 112 is transmitted to a remote server to participate in a federated learning framework in which global machine learning models are iteratively improved without transmitting raw user sensor data, thereby preserving privacy while enhancing model accuracy.

In one embodiment herein, the storage module 108 serves as the storage component of the system 100, holding the executable instructions, as well as any data or information required by the processor 138 to perform its tasks. The data includes user inputs, system configurations, and any other relevant data needed for the system's operations. Through the communication between the processor 138 and the storage module 108, the system 100 is able to process the user inputs, access stored information, perform computations, and make decisions accordingly.

In some embodiments, the primary wearable device 102 includes a voice interface 118, a control panel 122, and a power supply 140. The voice interface 118 is configured to deliver spoken coaching instructions. The control panel 122 comprises a plurality of buttons 124 that is configured to allow a user to control system operations. The power supply 140 is configured to power the primary wearable device 102. The primary wearable device 102 may further include one or more input/output ports 142, a wireless communication circuitry 144, and a visual LED indicator 146 configured to provide immediate feedback regarding exercise performance.

In some embodiments, the power supply 140 comprises a rechargeable battery configured for portable operation. In alternative embodiments, the primary wearable device 102 may be powered through a direct electrical connection using a corded power supply. The system 100 may support hybrid configurations allowing automatic switching between battery-powered and externally powered operation.

In certain embodiments, the processor 138 may comprise a heterogeneous processing architecture including one or more of a central processing unit (CPU), graphics processing unit (GPU), neural processing unit (NPU), tensor accelerator, digital signal processor (DSP), or application-specific integrated circuit (ASIC) configured to accelerate machine learning inference and motion analysis operations. The processing functions of the plurality of modules 104 may be distributed across dedicated hardware accelerators to reduce latency and power consumption during continuous exercise monitoring.

In alternative embodiments, the primary wearable device 102 may be implemented as a head-mounted device, wrist-worn device, chest-mounted module, smart garment integration, belt-mounted controller, or distributed multi-node wearable system in which multiple wearable components collectively perform sensing and processing operations while communicating through wireless communication circuitry 144.

In one embodiment herein, the system 100 further includes a locator module configured to assist in identifying the physical location of the primary wearable device 102 in cases of loss or misplacement. The locator module may utilize wireless signaling, sound emission, or communication with external devices to indicate the primary wearable device's 102 location.

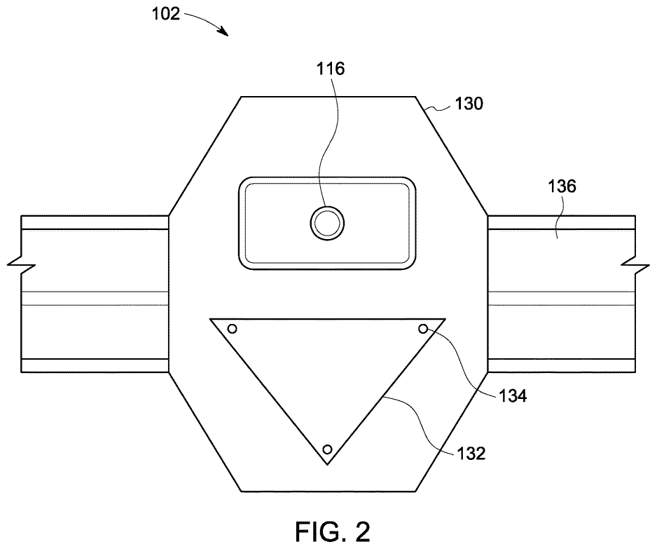

In one embodiment herein, the detection module 106 is configured to identify one or more fitness equipment located in a surrounding physical environment of the user using at least one scanning unit 116 (shown in FIG. 2). The scanning unit 116 can be, but not limited to, a visual camera, an RFID reader, a barcode scanner, a proximity detector, and thereof. The scanning unit 116 may scan identifying characteristics of the fitness equipment, such as shape, markings, embedded tags, or spatial location, which are then processed to determine the equipment type. This identification capability allows the system 100 to dynamically associate relevant exercise routines with the detected fitness equipment, thereby automating and personalizing workout planning.

In some embodiments, the scanning unit 116 operates by detecting identifying characteristics of one or more fitness equipment using one or more scanning technologies including visual camera recognition, radio frequency identification (RFID) detection, barcode scanning, or proximity sensing. Upon activation of an equipment scanning feature, the detection module 106 identifies the fitness equipment and communicates the detected equipment type to the processing module 110.

The processing module 110 then retrieves a plurality of exercise routines associated with the identified fitness equipment from the exercise database stored within the storage module 108. The user may then select a target muscle group or training objective via the control panel 122 and buttons 124 to refine the exercise recommendations.

In some embodiments, the exercise database stores each exercise routine as a structured data record including fields comprising an equipment type identifier, a target muscle group identifier, movement classification parameters, biomechanical reference thresholds, and instructional media pointers. The processing module 110 queries the database using equipment identifiers generated by the detection module 106 to retrieve exercise routines compatible with the detected fitness equipment.

In one embodiment herein, the storage module 108 is configured to maintain a database of exercise routines, which are categorized and organized into equipment type (e.g., dumbbells, cable machines), targeted body part (e.g., chest, hamstrings), training mode (e.g., resistance, cardio, stretching, plyometrics), and intensity level (e.g., beginner, intermediate, advanced). The storage module 108 may also include metadata such as form cues, instructional media, muscle group highlights, and substitution tags. This structured classification allows for rapid retrieval and personalized filtering of exercise routines based on user needs and detected context.

In one embodiment herein, the processing module 110 is configured to function as a core computational unit of the system 100. The processing module 110 may retrieve a set of relevant exercise routines from the storage module 108 upon identification of the fitness equipment by the detection module 106 and input from the user. The exercise routines are selected based on correlations among the detected equipment type, user-selected preferences such as training mode or targeted body part, and prior usage history or fitness goals. The processing module 110 may employ rule-based or AI-based logic to refine suggestions and sequence exercises optimally.

In one embodiment herein, the processing module 110 is further configured to receive user input indicating experiential issues encountered during exercise execution, such as discomfort, pain, or lack of interest. In response, the processing module 110 may communicate with the storage module 108 to identify and recommend one or more alternate exercises that target the same muscle groups or achieve similar training outcomes. These alternate routines are classified under “similar” or “dissimilar” categories, thereby enabling the user to make real-time adjustments during workout sessions.

In certain embodiments, the processing module 110 executes a machine-learning model configured to classify exercise movements and equipment types. The machine-learning model may include a convolutional neural network trained using labeled datasets of exercise images and motion sensor recordings. The model receives input data from the scanning unit and the tracking module 112, including visual image frames and inertial sensor signals, and generates output predictions identifying the detected fitness equipment and evaluating user posture during exercise execution.

In certain embodiments, the machine learning model comprises a convolutional neural network trained using a dataset comprising labeled exercise images and inertial sensor recordings representing different exercise movements. The dataset may include skeletal joint position labels, equipment identifiers, and motion trajectories corresponding to correct and incorrect exercise forms. The neural network is trained using supervised learning techniques to classify detected equipment types and exercise motion patterns based on visual frames captured by the scanning unit and inertial sensor signals generated by the tracking module 112. During inference, the trained model receives sensor data streams and outputs exercise classification results and posture evaluation scores.

The machine learning model executed by the processing module 110 may comprise one or more of convolutional neural networks, recurrent neural networks, transformer-based architectures, probabilistic graphical models, reinforcement learning agents, decision forests, support vector machines, or hybrid rule-learning systems configured to evaluate exercise performance and equipment identification.

In certain embodiments, the processor 138 continuously updates user-specific biomechanical reference profiles using adaptive learning algorithms configured to adjust posture thresholds, tempo recommendations, and corrective feedback sensitivity based on historical performance trends.

In some embodiments, exercise classification may alternatively be implemented using rule-based decision logic in which predefined motion thresholds and equipment identifiers are used to determine the exercise type without machine learning models.

In some embodiments, the processing module 110 is configured to perform feature extraction on sensor data by computing orientation vectors, joint angles, velocity profiles, and acceleration magnitudes derived from the inertial measurement unit sensors. These features are processed by the machine-learning model to detect deviations from predefined exercise movement patterns stored in the storage module 108.

In some embodiments, the processing module 110 determines recommended exercises using a rule-based decision engine that maps detected equipment type, target muscle group, and user fitness level to a predefined exercise selection matrix stored in the storage module 108. In other embodiments, the processing module 110 may employ the machine-learning model trained on historical workout datasets to classify suitable exercises based on input variables including detected equipment type, user performance metrics, and selected training objectives.

In one embodiment herein, the tracking module 112 is configured to monitor and evaluate user's physical movements in real time throughout the workout sessions. The tracking module 112 may utilize one or more technologies such as, but not limited to, motion sensors, at least one inertial measurement unit such as gyroscopes and accelerometers, and computer vision-based pose detection systems, to assess body alignment, movement patterns, range of motion, and tempo. This data is continuously processed to detect deviations from ideal form or unsafe mechanics. The tracking module 112 may also support both real-time correction and post-exercise analysis, thereby enhancing training safety and effectiveness.

In one embodiment, the motion sensors are configured to generate motion data representing body segment orientation. The processor 138 processes the motion data to determine joint angles and movement trajectories. The processor 138 executes a pose analysis model that is configured to compare the monitored body movement against predefined biomechanical reference models stored in the storage module 108.

In certain embodiments, joint angles are calculated using orientation data derived from the tracking module 112 comprises inertial measurement units (IMUs) embedded within the primary wearable device 102. The IMU sensors generate three-axis accelerometer and gyroscope data representing body segment orientation. The processor 138 converts the sensor data into quaternion orientation values and maps the orientations to a skeletal body model to estimate joint angles including elbow flexion, shoulder rotation, and knee extension.

In certain embodiments, the tracking module 112 further comprises additional sensing components including electromyography (EMG) sensors, pressure sensors, force sensors, magnetometers, ultra-wideband positioning sensors, lidar sensors, depth cameras, radar sensors, or physiological monitoring sensors configured to detect muscle activation, applied force, spatial positioning, or biometric response during exercise execution. Sensor outputs are fused by the processor 138 to generate an enhanced biomechanical representation of user movement.

The scanning unit 116 may further include environmental sensing components configured to detect spatial layout characteristics, equipment proximity relationships, lighting conditions, or user orientation within an exercise environment, thereby enabling contextual adaptation of exercise guidance based on environmental constraints.

In some embodiments, the tracking module 112 calculates joint angles by processing motion sensor data and estimating body segment orientation. The system 100 compares the calculated joint angles and movement trajectories against predefined biomechanical thresholds stored in the storage module 108 to determine whether the user is performing the exercise with correct form.

In one embodiment herein, the output module 114 is configured to communicate actionable information to the user. The output module 114 may deliver instructional guidance, corrective feedback, motivational prompts, and substitute exercise options based on inputs from the processing module 110 and the tracking module 112. The output is presented in both visual and auditory formats, thereby ensuring accessibility across diverse user preferences and training environments. The integration of real-time feedback promotes learning, performance improvement, and injury prevention.

As used herein, “real-time feedback” refers to feedback generated and delivered within a latency period of less than 200 milliseconds following acquisition of motion sensor data.

In one embodiment herein, the voice interface 118 is enabled by the output module 114 to deliver personalized auditory instructions. The voice interface 118 may incorporate user-specific identifiers, such as addressing the user by name, and provides real-time form corrections, encouragement, and reminders (e.g., “tighten your core,” “keep your elbows in”). The voice prompts can adapt in tone and frequency based on the user preferences.

In one embodiment herein, the display interface 120 is enabled by the output module 114 to visually present contextual information related to the selected exercise routines. The contextual information includes step-by-step instructions, muscle group highlights, recommended sets and repetitions, tempo guidance, and video demonstrations.

In some embodiments, the display interface 120 allows the users to input commands, receive information, and control the system 100. The display interface 120 can be, but not limited to, a touch screen, a keyboard, a mouse, voice recognition modules, gesture recognition sensors, and virtual reality interfaces. The versatility of the display interface 120 ensures that the users can engage with the system 100 in a manner that is most intuitive and comfortable for the users, thereby catering to a wide range of user preferences and accessibility needs.

In some embodiments, the display interface 120 comprises an augmented-reality visualization system configured to overlay exercise guidance indicators, alignment markers, or trajectory paths within a user's field of view, thereby enabling spatially registered coaching feedback during exercise execution.

The output module 114 may further include a haptic feedback subsystem configured to generate vibration patterns or localized tactile signals corresponding to detected posture deviations or tempo corrections, enabling silent corrective guidance without visual or audio output.

In one embodiment herein, the display interface 120 and the control panel 122 are functionally distinct. The display interface 120 is configured to present visual information, including exercise routines, instructional media, and system prompts, and may optionally allow navigation through touch or gesture-based input. In contrast, the control panel 122 is configured to execute predefined command functions during active operation, including mode selection, exercise substitution, routine switching, and system-level controls. The control panel 122 enables rapid interaction without requiring navigation through visual menus, thereby enhancing usability during physical exercise.

In one embodiment herein, the control panel 122 with the plurality of buttons 124 is configured to allow the user to select from multiple instructional modes, which include tutorial playback prior to performing the exercise, real-time voice-guided feedback during execution, and post-exercise feedback summarizing form adherence, completion metrics, and improvement suggestions, thereby accommodating varying levels of user autonomy and learning preferences. The buttons 124 are further configured to allow the user to indicate discomfort, pain, or disinterest during an exercise, initiate exercise substitution options, select instructional modes, adjust workout intensity, or activate features such as voice guidance, warm-up routines, or projection display. These buttons 124 enable quick and intuitive interaction with the primary wearable device 102 during active workout sessions without requiring manual navigation through menus.

In some embodiments, the control panel 122 supports an extended set of functional commands accessible via the plurality of buttons 124. These commands include a voice-only mode enabling audio-based guidance without visual output, a substitution mode for selecting alternative exercises targeting the same muscle group, a transitional exercise mode for switching between body parts using the same equipment, a customized routine mode allowing user-defined workout sequences, a randomized routine generation mode, specialized training modes corresponding to predefined performance goals, user category modes including children and senior-friendly functional training, sport-specific training configurations, pre-workout tutorial mode, post-workout feedback mode, body composition analysis and nutritional recommendation activation, projection-based instruction activation, image and video capture control, media playback control, secondary wearable device connectivity control, alarm and reminder activation, voice interface customization including tone and feedback frequency, and a locator function configured to assist in identifying the physical location of the primary wearable device 102.

In some embodiments, the system 100 provides three distinct instructional modes selectable through the control panel 122. A tutorial mode is configured to provide instructional videos, diagrams, and explanatory text prior to exercise execution through the display interface 120. A real-time motion analysis mode is configured to provide live coaching feedback during exercise execution using voice prompts generated through the voice interface 118 and motion data captured by the tracking module 112.

A post-exercise feedback mode is configured to generate performance summaries and corrective suggestions after completion of an exercise session, which are presented through the display interface 120 and output module 114.

In one embodiment herein, the primary wearable device 102 also includes an initialization feature for conducting warm-up routines to support safe and effective training. The users may select a warm-up protocol tailored to specific body parts (e.g., shoulder mobility for upper-body days) or a generalized full-body routine. These warm-ups are retrieved from the storage module 108 and executed through the output module 114 to prepare the user for primary exercises.

In some embodiments, the primary wearable device 102 may include a projection module 126 that is configured to display interactive instructional content onto a nearby flat surface using laser projection or similar technology, thereby allowing group users or individuals to visualize movement demonstrations or receive synchronized prompts without reliance on handheld screens or head-mounted displays.

In certain embodiments, the projection module 126 comprises a projection lens assembly disposed on an external surface of a housing 130 of the primary wearable device 102. The projection lens assembly is configured to project visual instructional content onto a nearby surface. The projection module 126 may include a micro-lens, laser projection unit, or digital light projection system integrated within the housing 130, wherein the lens is oriented to enable forward or downward projection during use.

In certain embodiments, the projection module 126 includes a micro-laser projector configured to project visual exercise instructions onto nearby surfaces. The projector operates using low-power scanning laser technology capable of displaying instructional graphics or movement guidance patterns.

In one embodiment herein, the primary wearable device 102 is architected to operate without requiring external smartphones, third-party applications, and continuous network connectivity. All processing, instruction, and feedback mechanisms are integrated within the primary wearable device 102 itself, thereby ensuring usability in offline or restricted environments such as commercial gyms or remote workout settings.

In some embodiments, the primary wearable device 102 is configured to operate in diverse environmental conditions including indoor, outdoor, and aquatic environments. The housing 130 of the primary wearable device 102 and associated components may be constructed using water-resistant or waterproof materials to protect internal electronics from moisture, sweat, and water exposure. In certain embodiments, the system 100 is adapted for use during water-based activities including swimming, aquatic resistance training, or rehabilitation exercises. The tracking module 112 and the detection module 106 may be configured to operate under such conditions using sealed sensor configurations and waterproofed sensing elements, thereby enabling exercise monitoring and feedback in water environments.

In certain embodiments, the tracking module 112 is configured to adjust motion analysis parameters when operating in aquatic environments to account for water resistance, buoyancy effects, and altered movement dynamics, thereby ensuring accurate biomechanical assessment during swimming or water-based exercises.

In one embodiment herein, the system 100 further includes a secondary wearable device 128, which can be, but not limited to, a visor or glasses. The secondary wearable device 128 is wirelessly connected to the primary wearable device 102 for displaying instructional visuals directly within the user's field of vision and functions without the need for external connectivity or mobile applications. The secondary wearable device 128 enhances immersion and allows for a more focused and engaging workout experience.

In one embodiment herein, the primary wearable device 102 enables the users to create, store, and modify custom exercise routines within the storage module 108. The exercise routines may be built using prior performed exercises, training strategies such as supersets or pyramid sets, or user-defined goals. The users may edit, reorder, or substitute exercises within saved routines, thereby supporting progressive overload and adaptation over time.

FIG. 2 refers to a rear perspective view of the primary wearable device 102 of the system 100. In one embodiment herein, the primary wearable device 102 comprises the housing 130, which is a geometric, generally octagonal in shape, and integrates both the scanning unit 116 and an enclosure 132. The scanning unit 116 is configured for scanning the fitness equipment and tracking user movements. The enclosure 132 is securely affixed to the housing 130 via a plurality of fastening elements 134, such as screws or magnetic couplers. The enclosure 132 is configured to enclose and protect a plurality of auxiliary components (not shown) such as, but not limited to, power supply units, rechargeable batteries, signal processing hardware, and local storage. The primary wearable device 102 further includes adjustable straps 136 that enable the primary wearable device 102 to be comfortably worn on various parts of the user's body, such as wrist, shoulder, waist, or upper arm, depending on user preference or training application. This compact and ergonomic configuration allows for hands-free operation and unobtrusive use during dynamic exercise sessions.

In certain embodiments, the primary wearable device 102 further comprises a mounting interface (not shown) configured to enable the primary wearable device 102 to be secured in a stationary position. The mounting interface may include at least one of a clip mechanism, magnetic attachment, suction fixture, strap-lock system, or threaded mount compatible with external support structures. This configuration allows the primary wearable device 102 to operate in a non-wearable mode, including placement on gym equipment, walls, stands, or poolside surfaces, thereby enabling stable tracking and projection during specific activities.

In certain embodiments, when used in aquatic environments, the mounting interface enables the primary wearable device 102 to be secured to a fixed structure adjacent to a water body, including pool edges or support fixtures. This allows the system 100 to monitor user movements during swimming or water-based exercises using the tracking module 112 and an image capturing module 150, while maintaining stability and accuracy of data acquisition.

For example, when the scanning unit 116 detects a dumbbell positioned within the exercise environment, the detection module 106 identifies the equipment type as a free weight device. The processing module 110 retrieves a list of exercises associated with dumbbells from the storage module 108, including biceps curls, shoulder presses, and chest fly movements. The user selects a target muscle group using the control panel 122, and the system begins tracking the user's motion using the tracking module 112 to analyze elbow flexion angle during a biceps curl movement.

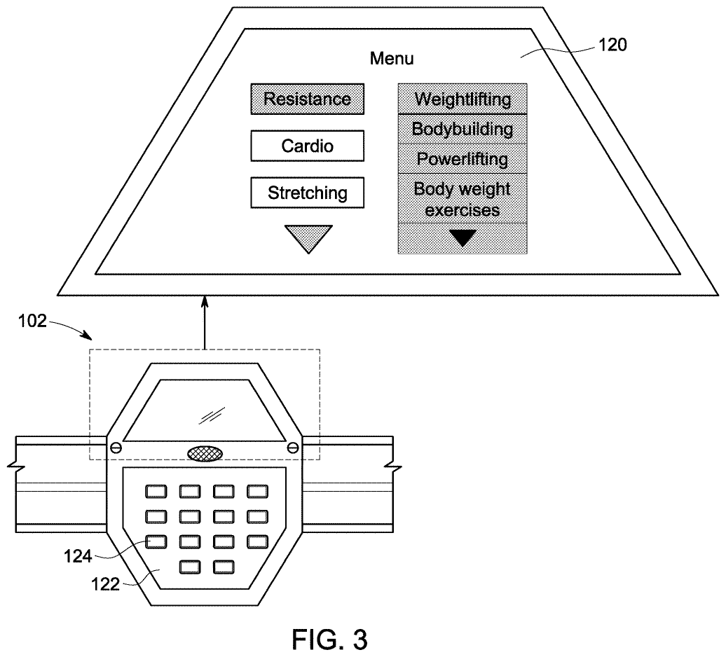

FIG. 3 refers to a perspective view of the primary wearable device 102 activated to initiate an operation of the system 100. First, the user powers on the primary wearable device 102 to initiate the operation of the system 100. Upon activation, the primary wearable device 102 activates the display interface 120 to prompt the user to access a customizable settings menu. Within this menu, the user can select among various exercise modes such as, but not limited to, resistance, cardio, stretching, and thereof, using the buttons 124 on the control panel 122. Each exercise mode is further subdivided into categories such as equipment-based or bodyweight-based exercises. Subcategories of resistance may include weightlifting, bodybuilding, powerlifting, body weight exercises (calisthenics), isotonic exercise, resistance bands, kettlebells, circuit training, isometric, plyometrics, isokinetic, yoga, pilates, strength and conditioning training, and thereof.

Subcategories of cardio may include high intensity interval training (HIIT), moderate intensity steady state (MISS), low intensity steady state (LISS), swimming, cycling, jump rope, running, stair climbing, dancing, jogging, kickboxing, walking, and thereof. Subcategories of stretching may include dynamic stretching, ballistic stretching, passive stretching, proprioceptive neuromuscular facilitation (PNF) stretching, active stretching, and thereof.

Once the activity mode is selected, the user can further refine the experience by choosing the intensity level, such as beginner, intermediate, and advanced. These settings tailor the exercise selection to match user capability, complexity of movements, and intended training goals. Next, the user is presented with an optional warm-up selection, where they may choose a warm-up routine targeted to specific body parts or opt for a full-body sequence. This warm-up protocol can be accepted or declined by the user based on their preferences.

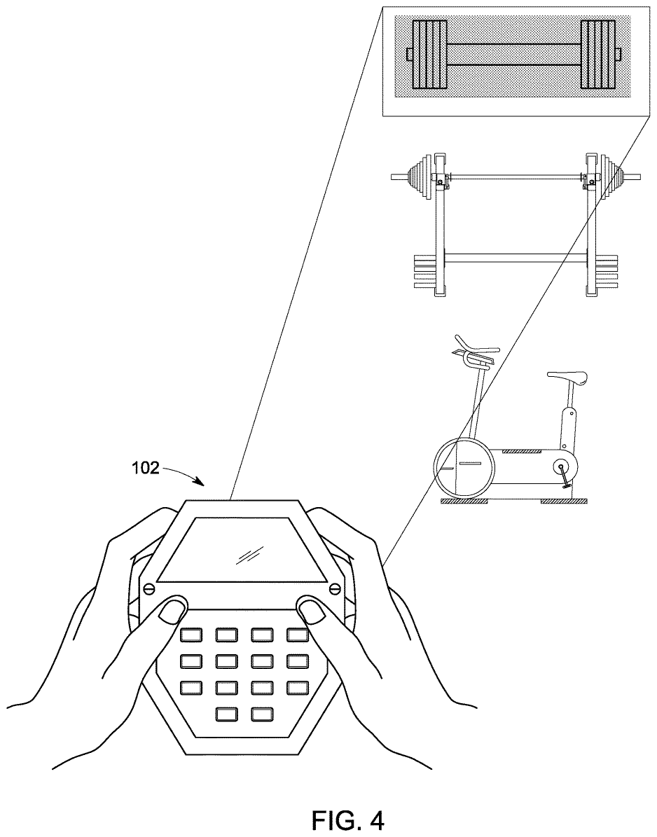

FIG. 4 refers to a perspective view of the primary wearable device 102 detecting at least one fitness equipment. In one embodiment herein, the user then activates the equipment scanning feature, which is enabled via the detection module 106 (shown in FIG. 1). The primary wearable device 102 uses the scanning unit 116 (shown in FIG. 2), which can be at least one of the visual cameras, the RFID reader, or the proximity detector, to scan at least one fitness equipment. Upon successful recognition, the primary wearable device 102 references its onboard exercise database in the storage module 108 (shown in FIG. 1) and retrieves a curated list of exercise routines associated with the identified fitness equipment using the processing module 110 (shown in FIG. 1). For example, if a dumbbell is scanned, the primary wearable device 102 retrieves a broad range of exercises, and the user is prompted to specify a target area such as biceps, shoulders, or chest for refinement.

In one embodiment herein, the operation of the system 100 follows a structured sequential protocol. Initially, the detection module 106 identifies at least one fitness equipment using the scanning unit 116. Upon successful identification, the processing module 110 retrieves a plurality of exercise routines associated with the detected equipment from the storage module 108. Subsequently, the user is prompted, via the control panel 122 or display interface 120, to select at least one target muscle group or training objective. This sequential arrangement ensures that exercise recommendations are first contextualized based on available equipment and thereafter refined based on user-specific preferences, thereby improving flexibility and user control.

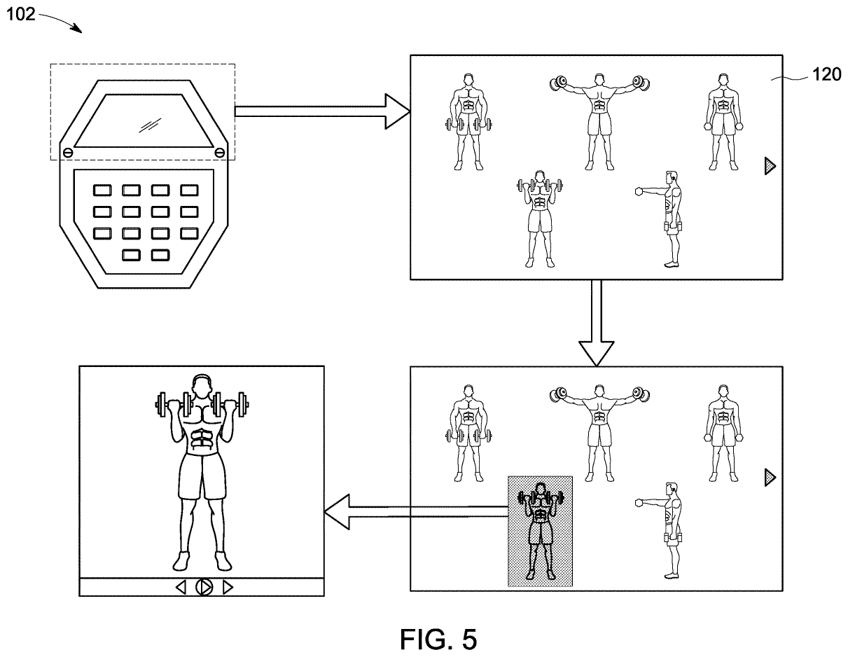

FIG. 5 refers to a perspective view of the primary wearable device 102 presenting exercise routines, choosing at least one routine, and delivering real-time visual guidance. In one embodiment herein, each retrieved exercise is displayed on the display interface 120, with at least 5 or more shown at a time via a scrollable interface. For each exercise, instructional data is accompanied by a muscle engagement visualization in which the targeted muscle group is highlighted in the instructional figure or video. The primary wearable device 102 allows the user to select an exercise and customize their guidance experience through three distinct modes. In pre-exercise mode, the user can view a video tutorial to learn the movement before performing it. In real-time mode, the primary wearable device 102 provides voice-activated guidance during the exercise, thereby offering immediate feedback to correct form and enhance performance. In post-exercise mode, the primary wearable device 102 scans the user's movements during the exercise and delivers detailed feedback afterward to support improvement.

Referring to FIG. 1, the tracking module 112 monitors the user's biomechanics through motion sensors and visual processing during execution. The system 100 detects errors in posture, alignment, tempo, and range of motion, and, depending on the selected mode, relays corrective prompts through voice output (e.g., “slow down your descent,” or “keep elbows close to your sides”). The system 100 may explain these prompts with brief justifications such as, “This helps reduce shoulder strain.”

If the user experiences discomfort, pain, or disinterest during an exercise, they can press a dedicated button 124 on the control panel 122 to activate the exercise substitution system. Upon activation, the system 100 promptly recommends alternative exercises targeting the same muscle group, categorized as either “similar,” which includes minor variations of the original movement, or “dissimilar,” which comprises entirely different exercises that achieve the same fitness objective.

In one embodiment herein, the system 100 further includes a transitional exercise mode configured to allow the user to switch from a first body part to a second body part while continuing to use the same identified fitness equipment. Upon receiving a transition command via the control panel 122, the processing module 110 retrieves alternative exercise routines associated with the same equipment but targeting a different muscle group. This functionality enables efficient workout flow without requiring re-scanning of equipment.

As used herein, a “similar exercise” refers to an exercise that targets the same primary muscle group and utilizes a comparable movement pattern or biomechanical motion as the original exercise. A “dissimilar exercise” refers to an exercise that targets the same primary muscle group but utilizes a different movement pattern or equipment type.

The primary wearable device 102 is capable of generating full workout programs automatically. The user may select a full-body, upper body, or lower body randomized routine composed of pre-selected exercises, with balanced targeting of major muscle groups. Alternatively, the user may build and save custom workout routines. The primary wearable device 102 offers templated formats supporting classic training structures, such as pre-exhaust, supersets, and pyramids, where the user can input their favorite or most effective movements. All exercise history and personal performance metrics, such as repetitions completed, weight lifted, sets executed, and personal bests, are stored by the storage module 108. The user may review this progress, track improvements over time, and save preferred workouts, exercises, and program formats for future sessions.

In one embodiment, the processing module 110 supports multiple workout routine generation methods, providing flexibility to users in creating personalized workout plans.

A goal-oriented routine generation mode allows users to input a fitness goal such as muscle gain, weight loss, endurance improvement, through the display interface 120. Based on this input, the processing module 110 analyzes the user's current fitness level, body composition, and training history, then generates a sequence of exercises designed to achieve the specified objective.

A customized routine mode allows users to manually select and arrange exercises from a list of available exercises retrieved from the storage module 108. Users can tailor the routine to their specific preferences by selecting exercises that target particular muscle groups or training styles such as strength training, HIIT, cardio. Once configured, the user can save the customized routine within the storage module 108 for future use, and the system 100 allows for easy retrieval and editing of saved routines.

A random routine generation mode allows the processing module 110 to automatically generate workout programs based on detected fitness equipment, the selected training mode such as resistance training, cardio, flexibility exercises, and available exercise categories stored in the storage module 108. The system 100 automatically selects and arranges exercises in a randomized sequence, offering users a new and diverse workout routine each time. This mode is ideal for users who seek variety or wish to avoid repetition in their workouts.

The processing module 110 interprets user interactions, exercise data, and equipment scanning results. The processing module 110 also enables dynamic learning by allowing the user to add new exercises by scanning movement patterns, instructional videos, or exercise names. Once scanned, the system 100 automatically classifies and stores these into the appropriate category in the database of the storage module 108.

The output module 114 delivers instruction and feedback through the voice interface 118 using the user's name for personalization, and also supports visual output, which may be presented on the display interface 120 or via external technologies. For immersive interaction, the projection module 126 projects large-format instructional visuals onto nearby surfaces. This is particularly advantageous for group cardio classes, and it synchronizes with voice instructions powered by embedded digital signal processors (DSPs) for high-quality audio clarity.

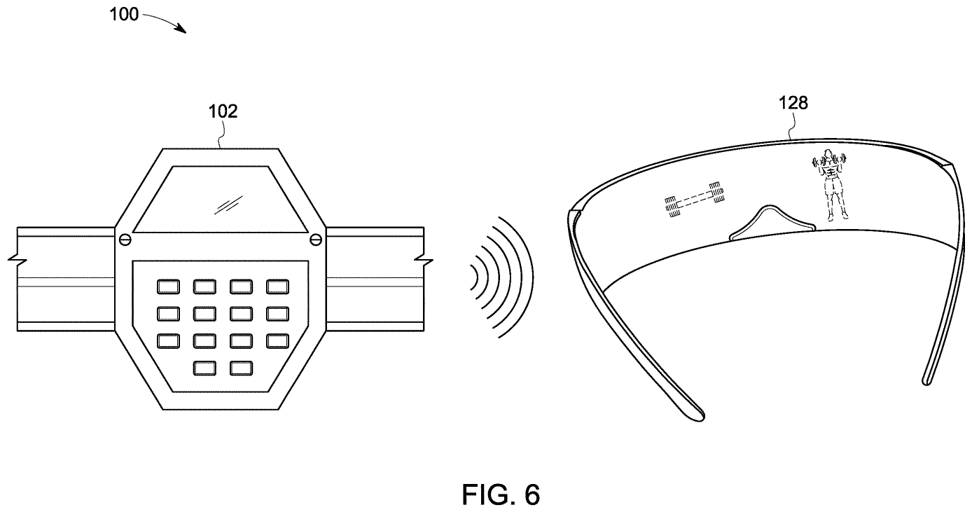

FIG. 6 refers to a perspective view of the secondary wearable device 128 of the system 100. In one embodiment herein, the primary wearable device 102 supports connection to the secondary wearable device 128, which can be visor-style smart glasses. The secondary wearable device 128 displays instructional visuals wirelessly without the need for Wi-Fi or an external app. The connection between the primary wearable device 102 and the secondary wearable device 128 may be established via at least one wireless communication protocol, such as Wireless Fidelity (Wi-Fi), Bluetooth, ZigBee, Z-Wave, Near Field Communication (NFC), Radio Frequency Identification (RFID), Ultra-Wideband (UWB), and thereof. This established connection allows the user to receive real-time form correction or movement demonstrations directly in their field of vision during the workout sessions.

In some embodiments, the primary wearable device 102 also includes a body composition analysis feature that evaluates the user's body type and outputs a diagnosis when selected. The system 100 prompts the user to mark the result as satisfactory or not satisfactory, and subsequently offers tailored recommendations such as gaining, maintaining, or losing weight, in accordance with the user's goals. To boost user engagement, the primary wearable device 102 delivers real-time motivational reminders during exercise, such as “stay focused on the mind-muscle connection,” “keep breathing steadily,” or “maintain a strong back arch during your bench press.” These audio cues, provided through the output module 114, promote proper technique and sustained mental focus, thereby enhancing the overall workout experience.

In certain embodiments, the body composition analysis feature utilizes bioelectrical impedance sensing integrated into the primary wearable device 102. Electrical impedance measurements are obtained by transmitting a low-level electrical signal through the user's body and measuring resistance values corresponding to body fat percentage and lean muscle mass. Alternatively, the system 100 may estimate body composition using image-based body shape analysis derived from captured images processed using computer vision algorithms.

In certain embodiments, the bioelectrical impedance sensing circuit includes two excitation electrodes and two sensing electrodes positioned on the primary wearable device 102 to contact the user skin. A low-amplitude alternating current is transmitted between the excitation electrodes while voltage differentials are measured by the sensing electrodes. The processor 138 calculates body impedance values using Ohm's law and maps the measured impedance values to body composition parameters including body fat percentage and lean muscle mass using stored calibration models.

In one embodiment, the system 100 further comprises a nutritional recommendation module 148 configured to generate dietary guidance based on user fitness goals and body composition analysis performed by the primary wearable device 102. The nutritional recommendation module 148 may generate recommendations including caloric intake levels corresponding to caloric surplus, caloric maintenance, or caloric deficit states.

The nutritional recommendation module 148 may further generate suggested macronutrient ratios to optimize performance and recovery, including carbohydrate intake for energy, protein intake for muscle repair and growth, and fat intake for hormone regulation and joint health. These ratios may be customized based on the selected training objective (e.g., strength training, endurance, fat loss) and adjusted over time based on progress and changing fitness goals. For example, the nutritional recommendation module 148 may recommend higher protein intake for muscle gain or a higher carbohydrate ratio for endurance training.

In one embodiment, the nutritional recommendation module 148 may analyze data such as the user's body fat percentage, lean muscle mass, activity level, and workout intensity to generate recommendations. These recommendations may include caloric intake levels tailored to the user's specific needs, including adjustments for caloric surplus, caloric maintenance, or caloric deficit based on the user's fitness goal (e.g., muscle gain, weight loss, or weight maintenance).

In certain embodiments, the nutritional recommendation module 148 operates by analyzing user activity data collected from the tracking module 112 and body composition measurements generated by the primary wearable device 102. Based on this analysis, the processor 138 calculates recommended caloric intake ranges and macronutrient distributions stored in the storage module 108.

In some embodiments, the primary wearable device 102 may feature input and output ports supporting USB drives, memory cards, and hard drives, thereby allowing the user to transfer media, such as music, which can be listened through earphones via an integrated headphone jack.

In one embodiment, the primary wearable device 102 further includes the image capturing module 150 configured to capture photographs and video recordings of workout sessions performed by the user. The captured media may include motion tracking overlays and performance metrics generated by the tracking module 112 and the processing module 110.

In one embodiment herein, the image capturing module 150 comprises a camera lens disposed on the housing 130 of the primary wearable device 102. The camera lens is positioned to capture images or video of the user's movement during exercise execution. The image capturing module 150 may include a front-facing or outward-facing camera, and may further include protective transparent coverings to prevent damage from environmental exposure.

In one embodiment, the image capturing module 150 is capable of capturing high-quality images and videos during the execution of exercises, providing visual documentation of the user's performance for later review or analysis.

In another embodiment, the image capturing module 150 may include a camera (such as a high-definition RGB camera, a depth camera, or an infrared camera) capable of capturing detailed images and videos of the user's movements during the execution of exercises. For example, the image capturing module 150 could include a front-facing camera mounted on the primary wearable device 102, positioned to capture the user's form and motion, or a sensor-based camera integrated with motion tracking capabilities. The captured images and videos provide visual documentation of the user's performance, enabling post-workout analysis and comparison with ideal form demonstrations or offering corrective feedback based on body alignment, posture, or exercise technique.

The recorded media may be stored locally within the storage module 108 and transferred through the input/output ports 142 or transmitted wirelessly via the wireless communication circuitry 144.

In one embodiment, the input/output ports 142 comprise USB ports or memory card slots, allowing for physical transfer to external devices like a computer or external storage. Alternatively, the recorded media may be transmitted wirelessly via the wireless communication circuitry 144, such as Bluetooth, Wi-Fi, or NFC, enabling seamless transfer to other compatible devices, including smartphones, tablets, or cloud storage services for later review or sharing.

In another embodiment, the wireless communication circuitry 144 can be, but not limited to, Local Area Network (LAN), Cellular Network, Wide Area Network (WAN), Intranet, Virtual Private Network (VPN), and wireless networks that use radio frequency (RF) or infrared (IR) technology to transmit data without the need for physical cables, thereby providing mobility and flexibility.

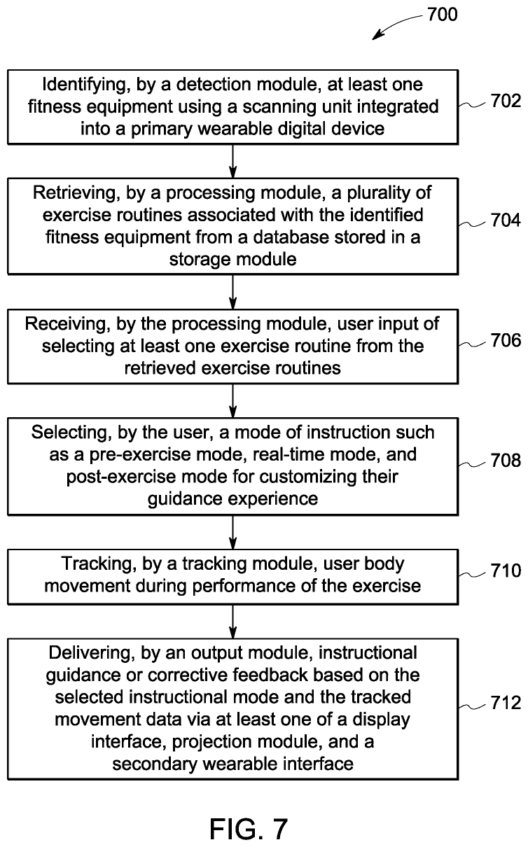

FIG. 7 refers to a flowchart 700 of a method for providing personalized exercise instruction using the system 100. First, at step 702, the detection module 106 identifies at least one fitness equipment using the scanning unit 116 integrated into the wearable digital device 102 of the system 100. At step 704, the processing module 110 retrieves the exercise routines associated with the identified fitness equipment from the database stored in the storage module 108. At step 706, the processing module 110 receives the user input of selecting at least one exercise routine from the retrieved exercise routines. At step 708, the user selects a mode of instruction such as a pre-exercise mode, real-time mode, and post-exercise mode to customize their guidance experience.

At step 710, the tracking module 112 tracks the user body movement during performance of the exercise. Further, at step 712, the output module 114 delivers the instructional guidance or corrective feedback based on the selected instructional mode and the tracked movement data via at least one of the display interface 120, the projection module 126, and the secondary wearable interface 128.

The proposed system 100 offers a significant advancement over conventional fitness systems by integrating a self-contained, AI-powered wearable device capable of autonomously delivering personalized exercise guidance, performance feedback, and workout customization. One of the foremost advantages is the device's ability to identify fitness equipment through the scanning unit 116, thereby enabling the users to instantly retrieve a curated list of exercises specifically tailored to the detected fitness equipment. This eliminates the need for manual search, mobile apps, or trainer dependency, thereby streamlining the workout planning process and enhancing user independence.

A key feature of the system 100 is its multi-modal instructional capability, which allows the users to select from three distinct modes of interaction, such as pre-exercise video tutorial, real-time voice-guided critique, or post-exercise analysis. This flexibility accommodates a wide range of user preferences, learning styles, and fitness levels. The integration of real-time motion tracking ensures that the users receive immediate feedback on form and technique, which is critical for preventing injury and reinforcing proper biomechanics. This hands-free, on-the-go feedback mechanism serves as a true digital personal trainer embedded within the primary wearable device 102.

The system 100 also excels in adaptability and personalization. The users can adjust the intensity level, select training modes across resistance, cardio, or stretching categories, and even customize or build entire workout routines using classic strategies such as supersets and pyramid sets. Additionally, the system 100 allows the users to flag exercises that cause discomfort or disinterest and provides smart substitution options categorized as “similar” or “dissimilar,” thereby maintaining workout consistency without compromising comfort or motivation.

Another advantage lies in the device's fully offline functionality. Unlike existing solutions that rely on smartphone integration, cloud connectivity, or mobile apps, the primary wearable device 102 operates as a standalone computing unit, thereby offering complete functionality without external dependency. This makes it ideal for environments where smartphone usage is restricted or internet access is unavailable. Further, the primary wearable device 102 supports wireless transmission of instructional content to the secondary wearable device 128 or the projection module 126, thereby enabling immersive, heads-up workout guidance without the need for Wi-Fi or third-party platforms.

In some embodiments, the visual LED indicator 146 is configured to provide immediate visual feedback regarding exercise performance. The LED indicator 146 emits a green color indicating correct exercise form when the tracking module 112 determines that the user's motion satisfies predefined biomechanical criteria, and emits a red color indicating incorrect form when improper movement patterns are detected. Additional colors may be used to represent intermediate performance states or warnings.

The primary wearable device 102 also enhances user engagement through features such as muscle group visual highlighting, voice-based personalization using the user's name, and reminder prompts for key fitness cues such as posture, breathing, and mental focus. Over time, the primary wearable device 102 tracks user progress, records performance metrics, and offers body composition assessments with actionable guidance based on whether the user desires to gain, maintain, or lose weight. Finally, the system 100 is designed with expandability and longevity in mind. The users can update the onboard database with new exercises by scanning movements, videos, or exercise names, thereby ensuring the system 100 evolves with the user's interests and trends in fitness. The ports for external media devices and earphone connectivity further enrich the user experience, thereby allowing music playback during workouts.

In a preferred embodiment, the primary wearable device 102 incorporates the tracking module 112 which is a nine-axis inertial measurement unit including a three-axis accelerometer, three-axis gyroscope, and three-axis magnetometer to enable accurate tracking of body movement during exercise execution. The processing module 110 executes a trained neural network model configured to classify exercise movements using sensor fusion data derived from the inertial sensors and image data captured by the scanning unit 116. The processor 138 performs sensor fusion of data received from the scanning unit 116 and the tracking module 112 to determine a skeletal movement model of the user during exercise execution.

In certain embodiments, the scanning unit 116 may operate in conjunction with external imaging devices positioned within the exercise environment, including ceiling-mounted cameras or equipment-mounted sensors. The processor 138 may receive image data from such external sensors through the wireless communication circuitry 144 and perform multi-source sensor fusion with inertial sensor data generated by the tracking module 112. This distributed sensing architecture allows the system 100 to maintain accurate exercise monitoring even when the wearable device 102 is partially occluded or positioned away from the user's primary movement axis.

In certain embodiments, the processing module 110 may execute machine learning inference locally on the primary wearable device 102 while periodically synchronizing model updates or exercise data with a remote computing platform via the wireless communication circuitry 144. Such hybrid edge-cloud processing enables continuous improvement of the classification model while maintaining low-latency exercise guidance during offline operation.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A system for providing exercise guidance to a user, comprising:
a wearable device configured to be worn by the user;
a scanning unit integrated with the wearable device, wherein the scanning unit is configured to detect and identify at least one fitness equipment located within an environment;
a processor disposed within the wearable device and operatively connected to the scanning unit, wherein the processor is configured to perform operations using motion sensor data and visual input data using a trained neural network,
wherein the artificial intelligence-based computational operations comprise:
storing a plurality of exercise routines associated with respective fitness equipment and target muscle groups using a storage module;
identify fitness equipment based on visual feature extraction or tag detection using data from the scanning unit;
retrieve one or more exercise routines associated with the identified fitness equipment from the storage module using a processing module;
monitor body movement of the user during execution of a selected exercise routine using a tracking module;
process, using fused inertial measurement unit (IMU) data and image data generated by the scanning unit, to compute the monitored body movement using a pose analysis model to determine a posture of the user;
compare the determined posture with predefined biomechanical reference data stored in the storage unit; and
generate exercise guidance as real-time corrective feedback signals comprise at least one of audio coaching instructions, visual exercise form correction indicators, and motion deviation alerts using an output module, wherein the feedback signals are generated based on deviation thresholds derived the predefined biomechanical reference data,
wherein the processor is configured to perform the identification, monitoring, comparison, and generation of exercise guidance in real time within the wearable device, thereby improving accuracy of posture detection and real-time biomechanical feedback accuracy and reducing processing latency within the wearable device.

2. The system of claim 1, wherein the scanning unit comprises at least one of a visual camera, a radio-frequency identification reader, a barcode scanner, and a proximity detector.

3. The system of claim 1, wherein the processing module is configured to retrieve exercise routines associated with a selected target muscle group.

4. The system of claim 1, wherein the tracking module comprises one or more motion sensors include an accelerometer and a gyroscope, wherein the one or more motion sensors are configured to generate motion data representing body segment orientation, wherein the processor is configured to process the motion data to determine joint angles and movement trajectories.

5. The system of claim 1, wherein the tracking module is configured to analyze at least one of body alignment, range of motion, and exercise tempo during execution of the selected exercise routine.

6. The system of claim 1, wherein the output module is configured to provide real-time corrective feedback during performance of the exercise routine.

7. The system of claim 1, wherein the output module is configured to provide instructional guidance in at least one of a pre-exercise tutorial mode, a real-time coaching mode, and a post-exercise feedback mode.

8. The system of claim 1, wherein the processing module is configured to recommend an alternative exercise based on user's discomfort and disinterest during performance of the exercise routine.

9. The system of claim 1, wherein the wearable device comprises a voice interface configured to deliver spoken exercise instructions.

10. The system of claim 1, wherein the wearable device operates without requiring an external mobile device and internet connectivity.

11. The system of claim 1, wherein the system comprises a nutritional recommendation module configured to generate dietary guidance based on user fitness goals and body composition analysis performed by the wearable device.

12. The system of claim 1, wherein the wearable device comprises a primary wearable device, and a secondary wearable device configured for coordinated operation.

13. The system of claim 1, wherein the wearable device further comprises an image capturing module configured to record user's exercise performance.

14. A method for providing exercise guidance using a wearable device, comprising:
detecting, by a detection module executed by a processing module of the wearable device, at least one fitness equipment within an environment of a user using a scanning unit of the wearable device;
retrieving, by the processing module, a plurality of exercise routines associated with the detected fitness equipment from a stored exercise database;
receiving, by the processing module, a user input of an exercise routine from the plurality of exercise routines;
enabling the user to select at least one of a plurality of instructional modes, which include a pre-exercise mode, a real-time mode, and a post-exercise mode;
monitoring, by a tracking module executed by the processing module, body movement of the user during execution of the exercise routine; and
providing, by an output module executed by the processing module, guidance or corrective feedback to the user based on the monitored body movement.

15. A wearable device, comprising:
a housing configured to be worn on a body of a user;
a scanning unit configured to identify fitness equipment based on visual feature extraction or tag detection;
a processor configured to retrieve exercise routines associated with the identified fitness equipment from a stored exercise database;
a tracking module configured to monitor movement of the user during execution of a selected exercise routine; and
an output interface configured to provide instructional guidance or corrective feedback to the user.

16. The wearable device of claim 15, wherein the wearable exercise guidance device comprises a display interface configured to present visual exercise instructions.

17. The wearable device of claim 15, wherein the wearable exercise guidance device comprises a control panel including a plurality of buttons configured to select exercise routines or instructional modes.

18. The wearable device of claim 15, wherein the wearable exercise guidance device comprises a projection module configured to project instructional content onto a surface.

19. The wearable device of claim 15, wherein the wearable device comprises a wireless communication module configured to transmit instructional content to a secondary wearable device.
